(19) United States
(12) Patent Application Publication
(10) Pub. No.: US 2008/0097342 A1
(43) Pub. Date: Apr. 24, 2008

(54) COVER/SHEATH FOR A NEEDLE AND METHOD OF RESHEATHING A USED NEEDLE

(76) Inventor: Vadim Alexander Gordin, Monroe, NJ (US)

Correspondence Address: DAVID L. KING, SR., 5131 N.E. COUNTY ROAD 340, HIGH SPRINGS, FL, 32643, US

(21) Appl. No.: 11/428,481

(22) Filed: Jul. 3, 2006

(51) Int. Cl. A61M 5/32

(52) U.S. Cl. 604/263

(57) ABSTRACT

A removable needle cover or sheath 10 is comprised of an elongated cover body 12 with a hollow central volume extending along the longitudinal length of the body 12 for sheathing a needle 30 and an open first end 16 for attachment to a hub 32 of the needle 30, wherein the cover body 12 has a longitudinal extending frangible seal portion 20 extending lengthwise from the open end 16 toward an opposite second end 14 on a wall of the cover body 12. The method of replacing a needle cover or sheath 10 on a used needle 30 is comprised of the steps of grasping the needle cover or sheath 10 in ones hand; squeezing the needle cover or sheath 10 between the thumb and forefinger on diametrically opposed sides of the cover or sheath 10; breaking a longitudinal frangible seal 20 on the cover or sheath 10 by application of the diametrically opposed squeezing to form a slot or slit; relaxing the squeezing force allowing the slot or slit to open; grasping the used needle 30 or device holding said needle 30 and laterally moving the used needle 30 through the slot or slit; and pushing the cover onto the hub 32 of the used needle 30. The steps of laterally moving preferably includes the step of tilting the needle 30 at an angle directing the tip of the needle away from the hand holding the cover or sheath 10 such the needle 30 near the hub enters the slot or slit first and thereafter pivoting or rotating the cover relative to said needle to pass through the slot or slit prior to pushing the cover 10 onto the hub 32.

### TECHNICAL FIELD

This invention relates to a cover or sheath used to cover a hypodermic needle more specifically it relates to a cover and a method of resheathing a needle after the needle has been used.

### BACKGROUND OF THE INVENTION

Hypodermic needles are commonly used to obtain blood samples, give injections and for infusions.

The hypodermic needle is often attached to the end of a syringe for giving injections. Such needles are commonly covered using a plastic covering that protects the user from inadvertent punctures. These covers typically provide a passageway for sterilization gasses to effectively sterilize the canula of the needle and the hub so that the patient can be assured that the injected needle will be sterile upon use. These covers typically are somewhat cylindrically shaped components that are injection molded and have an opening that attaches securely to the hub of the needle and yet provides an air passageway such that the gasses for sterilization can be effectively transmitted through the cover and to the component needle.

Alternatively, some needle covers have an opened end at the hub and an open end near the distal end or opposite end of the cover and that opening is generally plugged with a fiber material so that the component is breathable for allowing sterilization gasses to enter. In current usage these covers provide a means for easily removing the cover prior to use in a fairly effective and safe manner. Where the problem occurs is that these type covers require the nurse or practitioner to reinstall the cover by reinserting the needle into the circular opening of the cover after use. Recent studies have indicated approximately 30 percent of accidental puncture wounds occur from the use of hypodermic needles during the recapping or resheathing of the needle. This primarily occurs because the operator is directing the canula towards the hand holding the cover in such a fashion that a misguided needle can prick the hand causing an open wound. These open wounds commonly can result in a various number of diseases being transmitted to the health care provider. These diseases include herpes, streptococcus, staphylococcus, tuberculosis, malaria, syphilis, not to mention HIV and other potential risks that the practitioner would be most desirous in avoiding.

In U.S. Pat. No. 3,537,452 a cover for hypodermic needles is shown wherein an open slot or slit is provided along the longitudinal axis of the cover such that the resheathing of the used needle can occur by simply placing the needle parallel to the cap and slipping the needle into the slot or slit and then pressing the cover back onto the needle hub. Similarly U.S. Pat. No. 4,643,722 shows a similar needle cover that provides a removable closure strip that can cover the slot or slit, the strip being preferably tape that can be pulled off of the cover and thereby providing a slotted opening for the needle to slip into similar to that described in U.S. Pat. No. 3,537,452. Both of these devices provide a way of bringing the needle back into the cover in a manner that does not direct the pointed tip of the needle into any movement directly towards the hand holding the cover. While these devices are simple in their construction, the present invention provides an improvement over these devices such that the closure cover provides a superior way of providing a cover that will maintain the sterility of the hypodermic needle while at the same time provide a way of achieving a safer reattachment of the cover onto the hub of a used needle that will improve the ability of the practitioner to resheath the needle without the risk of punctures.

### SUMMARY OF THE INVENTION

A removable needle cover or sheath is comprised of an elongated cover body with a hollow central volume extending along the longitudinal length of the body for sheathing a needle and an open first end for attachment to a hub of a needle, wherein the cover body has a longitudinal extending frangible seal portion extending lengthwise from the open end toward an opposite second end on a wall of the cover body. The method of replacing a needle cover or sheath on a used needle is comprised of the steps of grasping the needle cover in ones hands; squeezing the needle cover or sheath on diametrically opposed sides of the cover or sheath; breaking a longitudinal frangible seal on the cover or sheath by application of the diametrically opposed squeezing to form a slot or slit; relaxing the squeezing force allowing the slot or slit to open; grasping the used needle or device holding said needle and laterally moving the used needle through the slot or slit; and pushing the cover onto the hub of the used needle. Preferably the needle is tilted at an angle such that the needle near the hub enters the slot or slit first and therefore the point of the needle is directed away from the hand holding onto the cover or shaft and thereafter pivoting or rotating the cover relative to the needle or vice versa to pass the remainder of the needle including the tip into the slot or slit prior to pushing the cover onto the hub.

### BRIEF DESCRIPTION OF THE DRAWINGS

These and further objects of the present invention will be more fully understood from the following description of the invention reference to the illustrations appended hereto:

FIG. 1 is a plan view of the needle and cover/sheath assembly.

FIG. 1A is a cross sectional view taken along lines 1A through 1A of FIG. 1.

FIG. 2 is a plan view of the cover/sheath of the present invention.

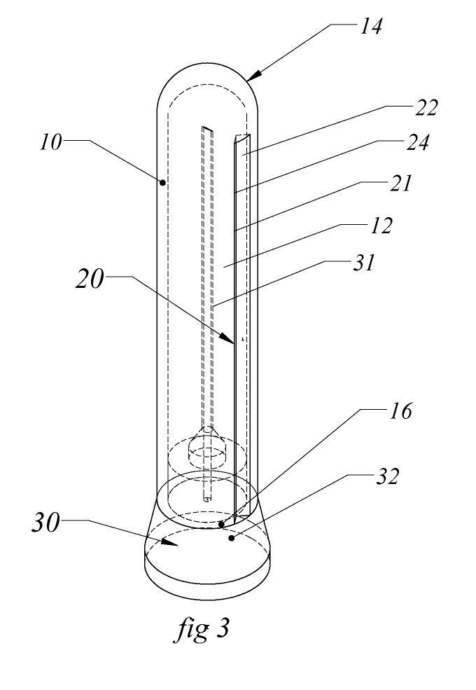

FIG. 3 is a perspective view of the cover/sheath of FIG. 2.

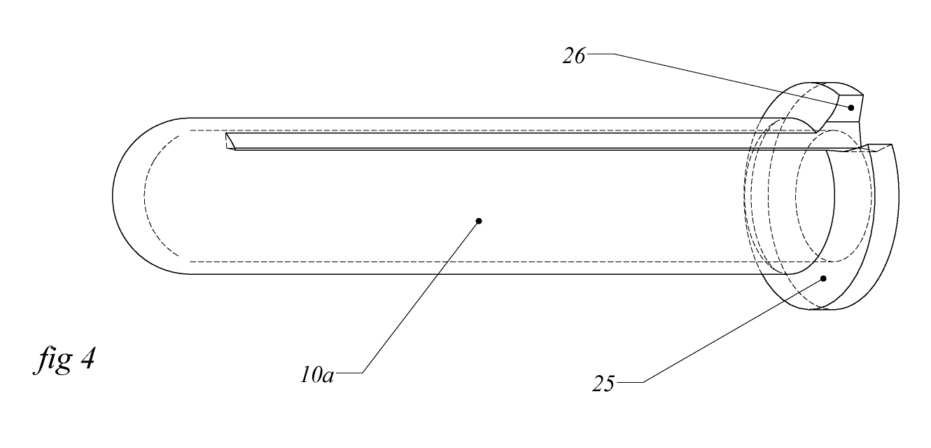

FIG. 4 is a perspective view of a flange base for the cover/sheath.

FIG.'s 5A through 5D are alternative embodiments of the present invention.

### DETAILED DESCRIPTION OF THE INVENTION

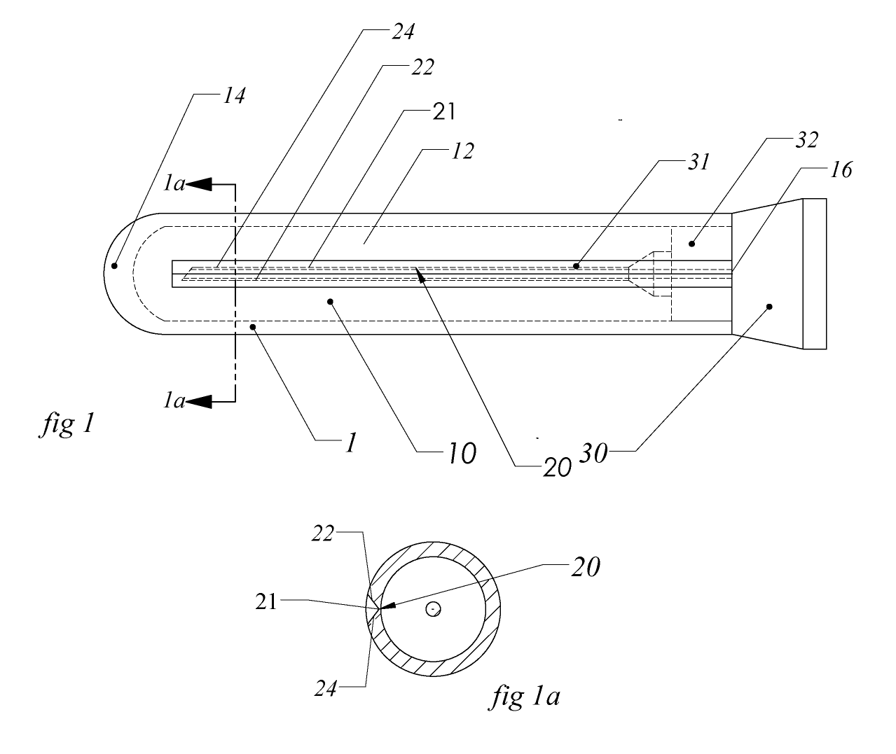

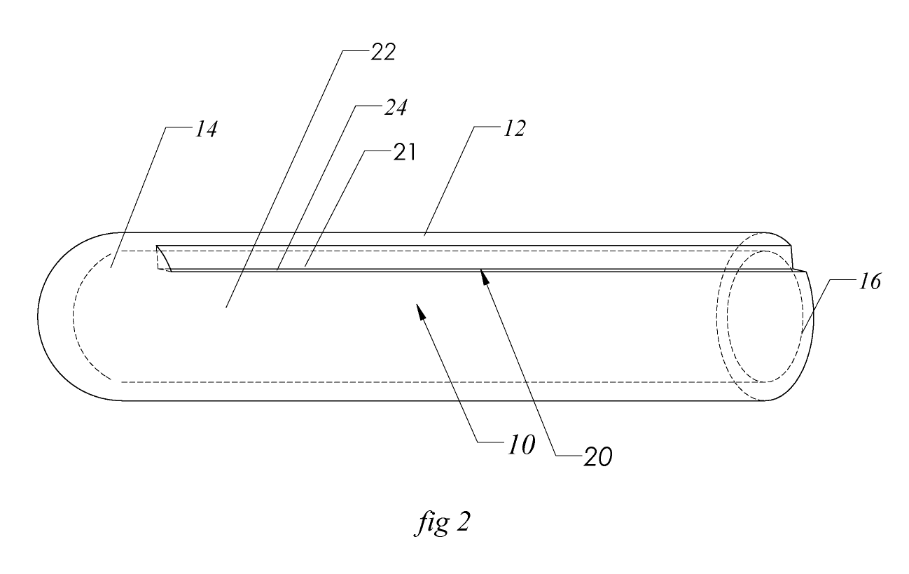

With reference to FIG. 1 a plan view of the cover/sheath for a needle is illustrated assembled to a needle 30. The cover/sheath 10 slips over a hub 32 of the needle assembly. The hub 32 has a metal canula or needle portion 31 extending therefrom. The needle portion 31 is securely affixed to the hub 32 in such a fashion that it cannot easily become dislodged. The needle 30 is easily attached to a syringe and or any type of device adapted to hold a needle. The needle 30 provides a passageway through the canula of the needle portion 31 to administer fluids or to take blood samples. A sharp tip is provided at the distal end of the needle 30 as shown. It is this tip that is particularly dangerous to the practitioner when administering medications or taking blood samples. Once used these devices need to be properly disposed of as they can be contaminated with various diseases which could cause infections if the practitioner is inadvertently stuck with the point of the needle portion 31 after being used and therefore contaminated.

The cover or sheath 10 as shown protects the needle portion 31 during normal storage and handling maintaining the needle in a sterile environment. Generally these covers are breathable such that sterilizing gasses can be passed internal of the cover 10 to ensure that the needle and hub portion 32 are effectively sterilized.

Syringes and other types of devices having hypodermic needles with such covers are generally packaged in a secondary outer package which may be paper or other type of packaging. Once opened in an aseptic fashion and the medical practitioner will remove the cover/sheath 10 exposing the needle 31. The needle 31 will then be injected into an injection site on an IV administration set or will be used to inject the patient directly in order to either administer medication and/or to withdraw blood samples. Once used the needle must be properly disposed of in such a fashion that it does not create a biological hazard or risk to the operator.

In the past these covers would simply be reinserted back over the hub through the opening at end 16 as illustrated. This provides an opportunity for the practitioner to stick himself or herself with the contaminated or otherwise used needle. This happens because the needle must move down the length of the cover and as such the operator by bringing the two components towards each other longitudinally is moving the pointed tip of the needle directly towards the operator's hand holding the cover. This creates an unacceptable risk for the practitioner, any slight distraction or bumping can cause the holder of the cover to jam the needle directly into his hand thereby causing the risk of contamination and infection to whatever disease may be on the needle tip.

As shown the cover has a frangible seal 20 provided along the longitudinal length of the cover from the open 16 extending towards the distal end 14 as shown. The end 14 as shown is a closed dome shape. The frangible seal 20 as illustrated in FIG. 1A has sloping surfaces 22 and 24 both inclined oppositely and coming to an apex or intersection of the surfaces 22, 24 along the cover wall as illustrated, wherein a thin breakable seam 21 has been formed at this apex or intersection of the two sloped surfaces 22, 24. This breakable seam 21 preferably has a very thin thickness such that it can be easily fractured. Depending on the cover material the thickness may be only a few thousandths of an inch thick. Once fractured the breakable seam 21 creates a slotted opening along the entire length of the frangible seal 20 as shown. The cover 20 has a body 12 that extends from the opening 16 to the closed end 14. The frangible seal 20 extends from the opening 16 towards the end 14, however, need not cover the entire length of the body, but preferably covers a length sufficient to accommodate the needle 30 to which it is attached.

The preferred method of using this cover/sheath 10 and 30 as a needle assembly is to remove the cover 10 from the needle hub 32 in an aseptic fashion and then using the needle 30 for whatever medical purpose is dictated, and then once used, resheathing the needle 30. This resheathing is accomplished by grasping the cover or sheath 10 and squeezing on two diametrically opposed sides such that the body 12 of the cover 10 is squeezed in such a fashion that the breakable seam 21 is fractured. This breakable seam 21 upon squeezing of the cover body 12 will fracture along the entire length of the seam 21. Once fractured a releasing of the pressure on the diametrically opposed sides of the cover 10 allows the frangible seal 20 to open along the breakable seam 21 forming a slot or slit in such a fashion that a needle 30 can be inserted back into the cover 10. Preferably the needle 30 is tilted at a slight angle such that the canula or metal portion 31 of the needle 30 closest to the hub 32 will enter the cover 10 first then by a slight pivotable motion or rotation the entire metal portion 31 of the needle 30 will slip easily into the slot or slit formed along the frangible seal 20. During this rotation it is important to note that the needle tip is never brought toward the hand holding the cover 10 in such a fashion that it could possibly contact the hand holding the cover because it will be tilted at an angle away from the hand prior to locking the portion 31 nearest the hub 32 into the slot or slit and as rotated down towards the hand the needle is secured and guided by the surfaces 22 and 24 such that it cannot slip out of the slot or slit and therefore there is virtually no risk that the user can puncture himself with the tip end of the canula.

The inventive cover/sheath 10 permits the needle 30 to enter along the longitudinal side of the cover/sheath once the metal portion 31 of the needle 30 is fully entered into the cover 10, the operator will then push the cover 10 back onto the hub portion 32 in such a fashion that the cover 10 is securely reattached. In this fashion the end 16 is designed to provide sufficient circumferential grip around the hub 32 even though a slot or slit exists in the area surrounding the hub 32, nevertheless the cover 10 will maintain adhesion to the needle hub 32 and will not become easily dislodged; similar to a split washer gripping a shaft.

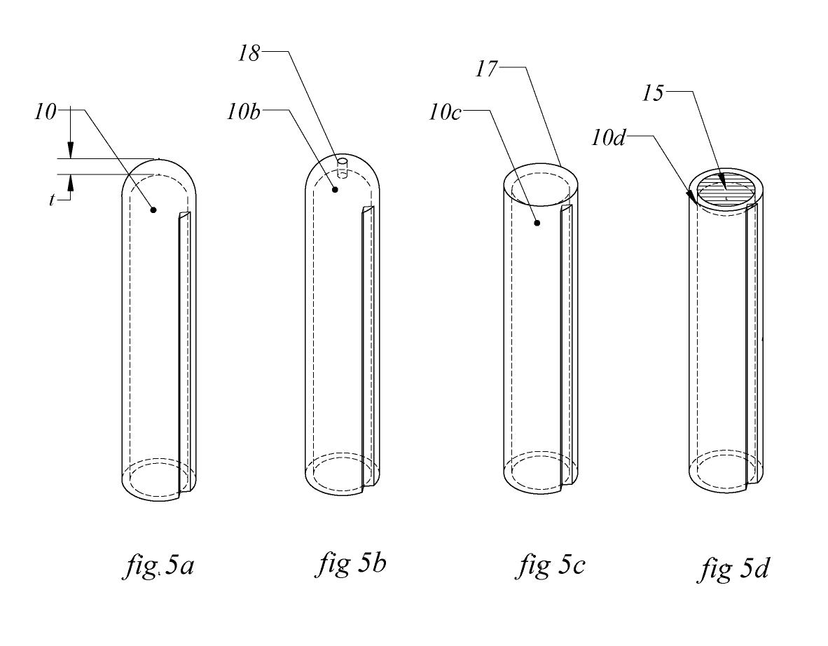

As shown in FIG. 4 at the hub end 16 the cover 10a may include a flange 25, the flange 25 provides a circular surface around the cover 10a up to the area where the frangible seal 20 occurred. At this point the flange 25 terminates in such a fashion that it has flared end 26 that follows the angular surfaces 22, 24 in such a fashion that upon breaking the seam 21 the flange 25 will not interfere with the fracture, furthermore, this type of flange 25 provides a mechanical stop so that the hands and fingers cannot slip past the cover 10a upon assembly or reassembly of the cover to the needle 30.

With reference FIGS. 5a-5d, alternative embodiments of the cover, 10, 10b, 10c and 10d with a frangible seal are shown.

With reference to FIG. 5b-5d, alternative end closures of the cover are shown. In the first embodiment the end closure is a sealed dome, the radius of curvature are different to provide a thickness (t) at the top of the dome. Alternatively the end closure of 5b can be an opening or hole 18 as shown in 5b which is breathable such that sterilization can be provided through this open end or hole forms a passage. In FIG. 5c at the end 14 has a flat sealed top 17. In FIG. 5d the end is open such that the cover 10d when formed as an extrusion has the frangible seal 20 extending the entire length of the cover 10d and a breathable plug 15 is inserted at the distal end, the plug end 15 is preferably made of synthetic fiber or cotton. These and various alternative configurations can be provided for any needle cover or sheath and still be within the scope of the invention provided that the cover/sheath has a longitudinally extending frangible seal 20 as illustrated along the body of the cover/sheath 10.

As illustrated the preferred embodiment covers 10, 10a, 10b and 10c an injection molded component preferably made of polyethylene, polypropylene or similar plastic material that can be easily processed and is suitable for creating a fracture seam along the side of the cover/sheath assembly.

Alternatively the cover/sheath can also be provided such that the distal end 14 is tapered smaller than the end having the needle hub 32 in such a fashion creating a truncated conical shaped cover assembly. Nevertheless the slot or slit formed at the frangible seal must be of sufficient width or sufficiently compliant if a slit to easily open upon needle pressure that it can easily accept the needle 30 using the preferred methodology of reassembly once the frangible seam and break line is broken.

Alternatively the cover 10d as shown in FIG. 5d can be an extruded component in such a fashion that it has a preferably tubular shape although any particular cross section can be used as long as the extrusion provides the breakable seam 20 extending along the entire length as illustrated in such a fashion the extruded component preferably has both ends open wherein the distal end 14 can be plugged using a fiber plug 15 or other means for sealing that end.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

1. A removable needle cover or sheath comprising:
an elongated cover body with a hollow central volume extending along the longitudinal length of the body for sheathing a needle and an open first end for attachment to a hub of a needle, wherein the cover body has a longitudinal extending frangible seal portion extending lengthwise from the open end toward an opposite second end on a wall of the cover body.

2. The removable needle cover or sheath of claim 1 wherein the frangible seal portion has a breakable seam, the breakable seam being responsive to a squeezing force on the cover body wherein the frangible seal portion opens along the length to provide an entry slot or slit for lateral resheathing of the needle in the removable needle cover.

3. The removable needle cover or sheath of claim 2 wherein the frangible seal portion has a pair of opposably inclined longitudinal extending surfaces extending to the breakable seam on the wall of the cover body to form a guide for lateral reinsertion of the needle.

4. The removable needle cover or sheath of claim 1 wherein the second end is closed.

5. The removable needle cover or sheath of claim 1 wherein the cover body tapers inwardly from the open hub end toward the second end.

6. The removable needle cover or sheath of claim 1 wherein the needle cover or sheath is an injection molded part.

7. The removable needle cover or sheath of claim 1 wherein the needle cover body is a tubular configuration.

8. The removable needle cover or sheath of claim 1 wherein the second end is open.

9. The removable needle cover or sheath of claim 8 further comprises a plug for inserting in the open second end.

10. The removable needle cover or sheath of claim 9 wherein the plug is a breathable fiber.

11. The removable needle cover or sheath of claim 1 wherein the second end is a closed dome.

12. The removable needle cover or sheath of claim 1 wherein the tubular body is an extruded part.

13. The removable needle cover or sheath of claim 6 wherein the cover or sheath is a polyethylene injection molded part.

14. The removable needle cover or sheath of claim 1 wherein the cover body has an external flange open at the frangible seal for providing a pushing surface for replacing the cover onto the hub of the needle.

15. The method of replacing a needle cover or sheath on a used needle comprises the steps of:
grasping the needle cover or sheath in ones hand;
squeezing the needle cover or sheath on diametrically opposed sides of the cover or sheath using the thumb and forefinger;
breaking a longitudinal frangible seal on the cover or sheath by application of the diametrically opposed squeezing to form a slot or slit;
relaxing the squeezing force allowing the slot or slit to open;
grasping the used needle or device holding said needle with the other hand and laterally moving the used needle through the slot or slit; and
pushing the cover onto the hub of the used needle.

16. The method of claim 15 further comprises the step of:
tilting the used needle or device holding said needle such that the tip of the needle is oriented away from the hand holding the cover or sheath and whereby the portion of the needle nearest the hub enters the slot or slit first; and thereafter rotating the cover relative to said needle to pass through the slot or slit prior to pushing the cover onto the hub.
